(19) United States
(12) Patent Application Publication
(10) Pub. No.: US 2003/0054070 A1
(43) Pub. Date: Mar. 20, 2003

(54) Edible solvent extraction of carotenoids from microorganisms

(75) Inventors: Terry Lawrence Bridges, Kailua-Kona, HI (US); Walter Nordhausen, Kailua-Kona, HI (US); Miguel Olaizola, Kailua-Kona, HI (US); Pertti Walliander, Kantvik (FI)

Correspondence Address: Martin E. Hsia, P.O. Box 939, Honolulu, HI, 96808-0939, US

(73) Assignee: Aquasearch, Inc.

(21) Appl. No.: 09/949,231

(22) Filed: Sep. 7, 2001

(51) Int. Cl. A23L 1/30; C07C 13/19

(52) U.S. Cl. 426/73; 585/23; 585/240

(57) ABSTRACT

A process for extracting carotenoids from a carotenoid-containing starting material produced by microorganisms is described. Said starting material is admixed with edible solvent to effectuate the transfer of carotenoids. Separation of the carotenoid-enriched edible solvent is improved by the formation of a “cake”, composed of said carotenoid-containing particulate solids, and, as required, a certain quantity of suitable filtration aid to modify the cake's consistency. Mechanical aids accelerate the separation of the carotenoid-enriched edible solvent. Said mixture may be hydrated to aid the removal of solids and gums from the carotenoid containing edible solvent. The carotenoid-enriched edible solvent is filtered though said cake to reduce the particulate load including any undesirable microbial load. A counter-current process increases the carotenoid concentration of the extract. The carotenoid-enriched edible solvent can be used as an ingredient in human and animal foodstuffs and dietary supplements for the possible prevention and treatment of illnesses and diseases.

### TECHNICAL FIELD

The present invention describes processes for producing carotenoid-rich edible extracts and residual solids.

### BACKGROUND ART

Carotenoids are a family of naturally occurring compounds of characteristically yellow, orange, and red color. Carotenoids are naturally synthesized only by algae, bacteria, cyanobacteria, plants, and fungi. Animals, including humans, cannot synthesize carotenoids de novo. The presence of carotenoids in human and animal tissues and fluids is caused by dietary intake of carotenoids.

Carotenoids are powerful antioxidants. This means that a carotenoid molecule has the ability to interact with oxidants such as free radicals and neutralize them. Free radicals are produced during normal metabolic processes in all living organisms. Living organisms are also exposed to free radicals from the environment. Free radicals are chemically reactive molecules that can damage otherwise healthy tissue and cellular components. This type of tissue and cellular damage can give rise to cancer and other diseases. Because carotenoids can neutralize free radicals, they are believed to play a significant role in the body's defenses against free radical attack. Free radicals are associated with the onset and progression of aging and of a number of diseases (including cancer, arteriosclerosis, cataracts, and macular degeneration), and thus carotenoids are expected to have a protective function against those diseases. Other functions of carotenoids include provitamin A activity and enhancement of the immune system.

Because of their protective fraction, carotenoid supplementation of the diet is believed to be beneficial to human and animal health. Natural carotenoids can be obtained from a diet rich in frits and vegetables. However, the concentrations of carotenoids found in such foods are often relatively low. The diet of the average person does not include large enough quantities of such fruits and vegetibles to assure a high intake of carotenoids. Thus, carotenoid supplements are quickly becoming a popular form of carotenoid intake. Carotenoid supplements can be made artificially from petrochemicals or extracted using petrochemicals or supercritical fluids from fruits, vegetables, algae, bacteria, fungi, and animal tissues. However, carotenoid supplements produced without the use of petrochemicals may be more desirable to consumers, and their production may have less impact on the environment.

Until recently, methods used for the extraction of natural carotenoids from biological material involved not only the use of various non-edible solvent systems, but also a large proportion of solvent in relation to the compound of interest. Many require the use of petrochemical or chlorinated solvents (e.g., methanol, ethanol, isopropanol, butanol, isoamyl alcohol, dimethylsulfoxide, acetone, hexane, toluene, xylene, benzene, methylene chloride, chloroform, etc. (see for example European Patent 0,612,725 “Solvent extraction of beta-carotene”, U.S. Pat. No. 4,341,038 “Oil Products from algae” and U.S. Pat. No. 5,714,658 “Process for the extraction of carotenes from natural sources”, and patents referred to therein, which are herein incorporated by reference, some of which may be highly flammable or toxic.)) As a result, these non-edible solvents must be eliminated almost completely from the finished product to ensure their safety for the consumer. Such solvent-removal and re-circulation systems are expensive and require special precautions for worker safety and environmental protection. The last traces of undesirable non-edible solvents and impurities in these non-edible solvents are very difficult to remove from the concentrated extract and residual amounts may remain in the extract even after the bulk of such non-edible solvents have been removed. The process claimed in the present invention does not use non-edible solvents, thus eliminating the potential for such residues. This is an advantage, as consumers may prefer carotenoid supplements and ingredients free of non-edible solvent residues.

Recent inventions (e.g., U.S. Pat. No. 5,591,343 “Process for extraction of carotenoids from bacterial cells” and U.S. Pat. No. 5,120,558 “Process for the supercritical extraction and fractionation of spices”, which are herein incorporated by reference) using a supercritical fluid process have been adapted for the extraction of carotenoids. This process requires high-pressure equipment to obtain good solvating properties of the pressure-liquefied gases. Some compounds may require the addition of co-solvents, which may be difficult to control and remove from the final product completely, or may be environmentally undesirable. However, another disadvantage in comparison with the present inventions is the higher cost for the required high-pressure system. No high-pressure vessels, pipes, or explosion-proof facilities are necessary for the present invention.

U.S. Pat. No. 4,713,398 “Naturally-derived carotene/oil composition” and U.S. Pat. No. 4,680,314 “Process for producing naturally-derived carotene/oil composition by direct extraction from algae” have been granted for an extraction process that does not use chemical solvents to extract carotenoids from algae. That process, however, uses other chemicals (e.g., alum and ferric chloride) as processing aids. Furthermore, other chemicals are also added in the process to adjust the pH of the product. As in the case for the non-edible solvents, there is an inherent risk of undesirable chemical residues in the final product when using such a process. The process described in the aforementioned patents is also inherently inefficient. As described in the patents, the process uses wet algal material (estimated to be substantially more than 90 percent moisture by weight), which is brought into contact with oil for the purpose of extraction. The presence of such large quantities of water would stand as a partial barrier between the biological material and the extracting oil. Increased contact between the oil and the biological material containing the carotenoids (such as cellular membranes) would allow a more efficient transfer of carotenoids from the biological material into the oil.

U.S. Pat. No. 5,773,075 “High temperature countercurrent solvent extraction of capsicum solids”, U.S. Pat. No. 6,013,304 “High temperature countercurrent solvent extraction of herb or spice solids”, and U.S. Pat. No. 6,074,687 “High temperature countercurrent solvent extraction of capsicum solids”, which are herein incorporated by reference, have been granted to Todd for the counter-current extraction of herbs and spice solids using edible solvents. The inventor claims a series of high-temperature (130 to 450 degrees Fahrenheit) mixing and pressure filtration (6,000 to 30,000 pounds per square inch) steps to produce concentrated herbal and spice extracts. The inventor claims reduction of microbiological load by the use of such high temperatures. This process would be unsuitable for the extraction of heat labile carotenoids, which decompose when subjected to high temperatures.

### DISCLOSURE OF INVENTION

The present invention claims the extraction of heat-labile carotenoids from microorganisms without the use of high temperatures, which is in contrast to the prior art. The present invention also uses a principle of mechanically improving the efficiency of the separation of the carotenoid-enriched edible solvent and residual solids, which is different from the prior art. This principle is the formation of a “cake” of residual solids, composed of the original carotenoid-containing particulate solids derived from microorganisms, and, as required, a certain quantity of a suitable filtration aid (such as diatomaceous earth of appropriate particle size), added to modify the consistency and porosity of the cake in order to achieve optimum flow rates without compromising retention of particulates. This process allows two important advantages that are superior to the prior art: firstly, the possibility of repeatedly circulating the edible solvent to efficiently extract without the use of heat, the desired carotenoid from the carotenoid-containing particulate solids derived from microorganisms, (thus enriching the carotenoid content of the edible solvent), and, secondly, the improved retention of particulates (which as a consequence eliminates, without the use of heat, any undesirable microorganisms that may contaminate the carotenoid-containing edible solvent).

The aforementioned U.S. Pat. Nos. 5,773,075, 6,013,304, and 6,074,687 describe the use of a filter press in the counter-current extraction of herbs and spice solids using edible solvents and high temperatures. The present invention uses different mechanical means for the separation of the carotenoid-enriched edible solvent and residual solids. Suitable mechanical means must permit the formation of the above-described cake of residual solids composed of the original carotenoid-containing particulate solids derived from microorganisms, and, as required, a certain quantity of a suitable filtration aid of appropriate particulate size. Such mechanical means include a centrifuge fitted with a perforated bowl or a membrane filter press.

Membrane filter presses can also be used for the separation of carotenoid-enriched edible solvent and residual solids. The operating principle of a membrane filter press is similar to the perforated bowl centrifuge as a cake is formed behind a filter membrane through which the filtrate is passed. The main difference is the source of force and the fact that the cake may be compacted more than in the perforated bowl centrifuge. The compaction results in good recover of the carotenoid-enriched edible solvent and a very dry cake. However, flow rates may be reduced thus prolonging the separation process.

Microorganisms, including the astaxanthin-containing cysts of Hacmatococcus pluvialis, by example, frequently have tough cell walls. These cell walls resist extraction of cell contents, and cell rupture may be required to achieve biological or chemical availability. This cell rupture process may result in very fine cell debris, which then requires a superior separation process. Traditional pressure filtration may not remove the fine cell debris. Instead, the use of a perforated-bowl centrifuge, in which a cake is formed inside the fine mesh insert, results in better filtration and adequate removal of fine particulates. This process leaves a certain percentage of the edible solvent used for extraction in the residual solids. In order to achieve an acceptable extraction yield of the carotenoid of interest, multiple extractions may be necessary. Therefore the present invention uses a multitude of mixing and extraction steps, where edible solvent washes are used to obtain carotenoid-enriched extracts from a cake. Specifically, a counter-current process is used to increase the concentration of the desired carotenoid fraction and the carotenoid yield of the extraction process. To reduce the amount of residual particulates, including undesirable microorganisms, washing of the extract though the cake formed by the use of a filtration aid (e.g., diatomaceous earth) is applied instead of high temperature. Extraction targets of the present invention are natural carotenoids from microorganisms.

The invention described here has the following advantages over the prior art:

1. The extracting solvent is a natural edible solvent, not a non-edible solvent.

2. The moisture content of the starting material to be extracted is much reduced (preferably less than 10 percent moisture by weight and optimally less than 5 percent moisture by weight), allowing for substantially more contact between the starting material and the extracting edible solvent, thus making the process more efficient.

3. No undesirable chemical additives are needed to help in the extraction of the carotenoid-enriched edible solvents.

4. The edible solvent-based extracts can be enriched in carotenoid concentration in comparison to the carotenoid concentration found in the starting material through the application of the multi-stage counter-current process.

5. The microbiological load in the carotenoid-enriched edible solvent extracts is lower than that of the carotenoid-containing starting material, due to the filtration of the carotenoid-enriched edible solvent extract through the cake, which removes particulates including microbes.

6. The yield of the extracted carotenoid from the starting material is greater than 90 percent by weight when a sufficient number of extraction steps are applied.

7. The high yield of the extracted carotenoid from the starting material is achieved even at the low temperature of about 30 degrees Celsius.

8. Heating of the starting material or other components is not necessary, as temperatures above 80 degrees Celsius may cause loss of astaxanthin or other heat-labile carotenoids, and is thus undesirable.

9. The final product obtained with this process will thus be a truly natural product and of greater attractiveness to the consumer.

### BRIEF DESCRIPTION OF DRAWINGS

FIG. 1 shows a schematic illustration of the counter-current extraction process.

### BEST MODES FOR CARRYING OUT INVENTION

The cell-ruptured, dehydrated, and coarsely ground (particle size less than 2 mm) carotenoid-containing starting material is mixed with edible solvent (which may have already been enriched in carotenoid content). An inert filtration aid such as diatomaceous earth may be added to the mixture to modify the consistency of the cake. Manipulation of cake consistency to optimize extraction efficiency and flow rates is within the skill of the art. Said mixture is transferred to the perforated-bowl centrifuge equipped with a fine mesh insert of appropriate mesh fineness while the centrifuge is spinning at a rate sufficient to provide the centrifugal force required to form the necessary cake of the appropriate consistency on the fine mesh insert. Following the transfer of the carotenoid-containing starting material to the centrifuge, the speed of the centrifuge is increased sufficiently to force the carotenoid-enriched edible solvent through the cake thus effectuating the further transfer of carotenoid from the carotenoid-containing starting material to the carotenoid-enriched edible solvent, which is collected from the centrifuge until the surface of the cake begins to dry. The carotenoid-containing edible solvent is returned to the centrifuge for further enrichment in carotenoid content, and to remove residual particulates from the carotenoid-containing edible solvent. This process may be repeated. The carotenoid-enriched and particulate-reduced edible solvent is then saved for further processing.

The next step involves the transfer of carotenoid-enriched edible solvent of lower carotenoid concentration than in the previous step (e.g. counter-current wash fraction) to the centrifuge for further enrichment in carotenoid content. This step may be repeated several times with carotenoid-enriched edible solvents of decreasing carotenoid concentration. The final step involves the transfer of edible solvent free of carotenoids to the centrifuge and the collection of the final carotenoid-containing edible solvent from the centrifuge.

The centrifuge is stopped and the cake removed from the fine mesh insert in the perforated bowl of the centrifuge. Depending on the residual concentration of carotenoids in the cake, the cake may be re-mixed with edible solvent and the above steps may be repeated.

Once the carotenoid concentration of the residual solids achieves acceptable low levels, the cake may be removed and the process repeated with fresh cell-ruptured, dehydrated, and coarsely ground carotenoid-containing material, which can be mixed with carotenoid-containing edible solvent and the above steps repeated.

The repetition of above steps will increase the carotenoid concentration of the carotenoid-enriched edible solvents. This repetitive process is referred to as the counter-current extraction process.

The viscosity of the edible solvents decreases with increasing temperature. Lower viscosity improves flow-rates of the edible solvent during the extraction and separation process. However, carotenoids are heat labile. Therefore the temperature of components, which are in direct contact with the carotenoid containing material, should be kept below the temperature at which losses of carotenoids occur. The following examples are given to illustrate the present invention but are not to be construed as limiting.

### EXAMPLES

1. Cell-ruptured, dehydrated, and ground green alga (Haematococcus pluvialis) was admixed with about 50 percent by weight of an edible solvent (rice bran oil) and centrifuged in a counter-current extraction process involving the initial extraction followed by up to six (6) cake washing stages for each set of extractions, each using a Mettich centrifuge equipped with a perforated bowl. The perforated bowl was lined with a high thread-count linen cloth bag to aid in the retention of the cake formed by the residual solids. Diatomaceous earth was added to the mixture as a filtration aid at a rate of five percent of the total weight of the ground algal meal and rice bran oil. Extracts from the second through last stages were returned to the process for use in washing the next cake, thus increasing the carotenoid concentration of each subsequent oil extract. Table 1 shows the astaxanthin concentrations of the collected extracts and residual solids from three cakes using a counter-current process, with extracts from the second through last stages being returned to the process for use in washing of the next cake thus increasing the carotenoid concentration of each subsequent oil extract. In each extraction series, every wash except for the final wash was carried out using the previously carotenoid-enriched edible solvent, and only the final wash was conducted with fresh rice bran oil. All steps of this process were conducted at room temperature of about 20 degrees Celsius.

The astaxanthin yield of the collected extracts was about 90 percent of the amount of astaxanthin-containing starting material (Table 2). The amount of edible solvent recovered was also about 90 percent (Table 3).

TABLE 1
Astaxanthin concentration of oil extracts (percent of total weight)
using Mettich perforated-bowl centrifuge.

| Astaxanthin | | | |
|---|---|---|---|
| concentration | | | |
| (weight percent) | First cake | Second cake | Third cake |
| Starting material | 1.14 | 1.14 | 1.14 |
| 1^{st} supernatant | 1.20 | 1.23 | 1.16 |
| 1^{st} wash | 0.22 | 0.55 | 0.86 |
| 2^{nd} wash | 0.09 | 0.29 | 0.47 |
| 3^{rd} wash | 0.05 | 0.15 | 0.26 |
| 4^{th} wash | 0.05 | 0.10 | 0.19 |
| 5^{th} wash | | 0.08 | 0.12 |
| 6^{th} wash | | | 0.12 |
| Residual cake | 0.32 | 0.36 | 0.56 |

TABLE 2
Astaxanthin extract yield (gram) using Mettich perforated-bowl
centrifuge. Values in parenthesis represent the amount of
astaxanthin added to the supernatant from the previous extraction.

| Astaxanthin extract yield | | | |
|---|---|---|---|
| (gram) | First cake | Second cake | Third cake |
| Starting material | 10.45 | 10.53 | 10.62 |
| 1^{st} supernatant | 7.76 | 7.29 | 6.58 |
| 1^{st} wash | 0.86 | 2.23 (0.86) | 3.25 (2.23) |
| 2^{nd} wash | 0.31 | 0.91 (0.31) | 1.28 (0.91) |
| 3^{rd} wash | 0.18 | 0.49 (0.18) | 0.72 (0.49) |
| 4^{th} wash | 0.18 | 0.37 (0.18) | 0.60 (0.37) |
| 5^{th} wash | | 0.29 | 0.41 (0.29) |
| 6^{th} wash | | | 0.48 |
| Total astaxanthin extract | 9.29 | 11.58 (1.53) | 13.32 (4.29) |
| Extraction yield (percent) | 88.90 | 95.44 | 85.03 |

TABLE 3
Edible solvent yield (gram) using Mettich
perforated-bowl centrifuge.

| | First | First | Second | Second | Third | Third |
|---|---|---|---|---|---|---|
| | edible | cake | edible | cake | edible | cake |
| Edible solvent | solvent | edible | solvent | edible | solvent | edible |
| yield (gram) | wash | extract | wash | extract | wash | extract |
| Starting | 919.80 | | 922.00 | | 932.10 | |
| material | | | | | | |
| 1^{st} supernatant | | 646.98 | | 594.78 | | 563.30 |
| 1^{st} wash | 332.35 | 394.05 | 389.20 | 407.02 | 368.42 | 377.30 |
| 2^{nd} wash^{rd} | 353.67 | 356.18 | 351.10 | 317.38 | 277.84 | 270.10 |
| 3^{rd} wash | 349.35 | 354.29 | 349.60 | 323.33 | 284.12 | 280.00 |
| 4^{th} wash | 353.69 | 393.40 | 388.80 | 362.66 | 324.59 | 313.00 |
| 5^{th} wash | | | 341.16 | 382.00 | 342.72 | 337.20 |
| 6^{th} wash | | | | | 362.40 | 393.80 |
| Total | 2308.82 | 2144.90 | 2741.86 | 2387.17 | 2892.19 | 2534.70 |
| astaxanthin | | | | | | |
| extract | | | | | | |
| Edible solvent | | 92.90% | | 87.06% | | 87.64% |
| yield (percent) | | | | | | |

2. Cell-ruptured, dehydrated, and ground green alga (Haematococcus pluvialis) was admixed with about 50 percent by weight of an edible solvent (rice bran oil) and the carotenoid-enriched edible solvent separated from residual solids using a Larox PF0.1 membrane filter press. Five percent by weight of diatomaceous earth was added to the mixture of the ground algal meal and rice bran oil as a filtration aid. The residual solids were washed four times, each time using fresh rice bran oil. All steps of this process were conducted at room temperature of about 20 degrees Celsius. Table 4 shows the astaxanthin concentrations of the collected carotenoid-enriched edible solvent fractions and the residual solid cake.

TABLE 4
Astaxanthin concentration in edible solvent extract (percent
of total weight) using Larox Membrane Filter Press Model PF0.1

| | Astaxanthin |
|---|---|
| | concentration in oil |
| | extract |
| Starting material | 1.11 |
| 1^{st} supernatant | 1.40 |
| 1^{st} wash | 1.35 |
| 2^{nd} wash | 0.14 |
| 3^{rd} wash | 0.07 |
| Residual cake | 0.15 |

3. 12 kilograms cell-ruptured, dehydrated (about 4 percent moisture), and ground green alga (Hacmatococcus pluvialis) containing 3.1 percent astaxanthin by weight, was admixed with 9 kilograms of rice bran oil. 7.5 kilograms of water were added to hydrate the algal particles. Vigorous stirring continued for 2 hours. The suspension was transferred to a Robatel Model RC/DRC 40 VxR equipped with a 400 mm diameter solid bowl, at a rate of 400 milliliters per minute, and centrifuged at 3000 revolutions per minute (equivalent to 1459 times the force of gravity). All steps of this process were conducted at temperatures of about 25 degrees Celsius. The recovered clarified oil produced by this single extraction step contained 1.65 percent of astaxanthin by total weight. The over-all yield of astaxanthin from the starting algal meal to the clarified oil was 38 percent by total weight. The residual cake contained 1.16 percent astaxanthin by total weight.

4. 6.6 kilograms of a mixture of cell-ruptured, dehydrated (about 4 percent moisture by weight), and ground green alga (Haematococcus pluvialis), rice bran oil (approximately 30 percent by weight), and water (approximately 30 percent by weight) containing 1.18 percent astaxanthin by total weight was admixed with 11.0 kilograms of rice bran oil and 0.4 kilogram diatomaceous earth as a filtration aid. The astaxanthin concentration of the resulting mixture was 0.44 percent by total weight. Diatomaceous earth was added at about 2 percent by weight to each washing oil fraction to maintain appropriate porosity of the cake. The suspension was transferred to a Robatel Model RC/DRC 40 VxR equipped with a 400 millimeter diameter perforated bowl, lined with a filter bag of 50 micrometer mesh, and centrifuged at 2400 revolutions per minute (equivalent to 934 times gravity). All steps of this process were conducted at temperatures of about 25 degrees Celsius.

The collected supernatant weighed 8.0 kilograms and contained 0.56 percent astaxanthin by total weight. The astaxanthin yield from the feed-mixture to the supernatant was 57 percent. The cake washing was conducted in counter-current mode using five aliquots of fresh oil. Wash oil fractions, and oil collected from the final cake-drying centrifugation step, were collected, providing a total of 19.5 kilograms oil containing 0.11 percent astaxanthin by weight, which is equivalent to 28 percent by weight of the astaxanthin content of the original mixture.

The final centrifuged cake weighed 6.6 kilograms and contained 0.14 percent astaxanthin by weight. The astaxanthin loss through the cake from initial mixture was 12 percent. The cumulative (as described in Examples 3 and 4) astaxanthin yield from the cell-disrupted and dehydrated Haematococcus pluvialis meal to the edible oil extract was 92 percent.

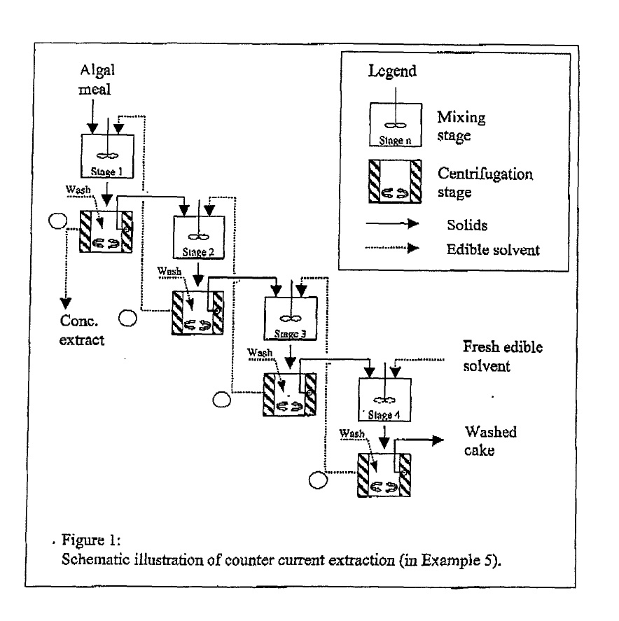

5. In order to increase the concentration of carotenoids in edible solvent extracted from the carotenoid-containing particulate solids, the carotenoid-containing edible solvent (collected in Example 4) was used to re-suspend residual solids that had previously been extracted with an edible solvent, as illustrated in FIG. 1. The astaxanthin concentration of the supernatant increased with each extraction. The carotenoid-containing edible solvent fractions of Example 3 were used in a counter-current fashion to wash the cake after each preceding supernatant carotenoid-containing edible solvent had been collected. During each stage the most concentrated wash was combined with the supernatant and an equal amount of fresh edible-solvent was added as last counter-current wash. The typical amount of each carotenoid-containing edible solvent wash was about 17 percent by weight of the carotenoid-containing starting material. All steps of this process were conducted at temperatures of about 25 degrees Celsius.

The concentration of supernatant increased gradually with each subsequent extraction step, starting from 0.56 percent astaxanthin by total weight (in Example 3) (A in FIG. 1), and increasing to 0.93 percent, 1.13 percent, and, finally, 1.28 percent astaxanthin by total weight (B, C and D in FIG. 1).

The residual astaxanthin concentration of each residual cake was about 0.15 percent astaxanthin by total weight, thus maintaining astaxanthin yield for each step similar to that shown by EXAMPLE 3. A total of five wash steps was sufficient to accomplish this.

6. The counter-current extraction process described in Example 5 provides a method to increase the concentration of astaxanthin of the edible oil extract, without sacrificing the astaxanthin yield of the extraction process.

Rice bran oil containing astaxanthin was admixed with cell-ruptured, dehydrated (about 4 percent moisture), and ground green alga (Haematococcus pluvialis) containing 4.0 percent astaxanthin by total weight. All steps of this process were conducted at temperatures of about 25 degrees Celsius. Applying a five step counter-current process resulted in the collection of supernatant carotenoid-enriched edible solvent fractions containing 1.48 percent, 2.88 percent, 4.92 percent, 5.76 percent, and 6.01 percent astaxanthin by total weight, respectively.

7. 158.7 kilograms of cell-ruptured, dehydrated (about 4 percent moisture), and ground green alga (Haematococcus pluvialis) containing 2.9 percent astaxanthin by total weight, was admixed with 174.9 kilograms of edible solvent (rice bran oil). Diatomaceous earth was added as a filtration aid to maintain the appropriate cake porosity. No water was added. 28.2 kilograms of the suspension was transferred to a Robatel Model RC/DRC 40 VxR equipped with a 400 millimeter diameter perforated bowl, lined with a filter bag of 50 micrometer mesh, and centrifuged at 2400 revolutions per minute (equivalent to 934 times gravity). The supernatant from the first extraction had an astaxanthin concentration of 1.35 percent by total weight. The residual solids cake in the centrifuge bowl was washed using the counter-current process in five successive steps, each with decreasing astaxanthin concentration of the carotenoid-enriched edible solvent. The last extraction was conducted using fresh edible solvent. Each addition of edible solvent to the residual carotenoid containing solids consisted of about 4 kilograms. All steps of this process were conducted at temperatures of about 25 degrees Celsius. The astaxanthin concentration in the carotenoid-enriched edible solvent fractions resulting from successive washes presented in Table 5 was similar to the data presented in Example 2.

TABLE 5
Astaxanthin concentration in edible solvent extract (percent of totid
weight) usiDg a ROBATEL centrifuge

| | Astaxanthin |
|---|---|
| | concentration |
| Sample | (weight percent) |
| Starting material | 1.38 |
| 1^{st} supernatant | 1.38 |
| 1^{st} wash | 1.21 |
| 2^{nd} wash | 0.99 |
| 3^{rd} wash | 0.85 |
| 4^{th} wash | 0.69 |
| 5^{th} wash | 0.51 |
| 1^{st} residual cake | 0.48 |
| Re-suspended 1^{st} cake | 0.33 |
| 1^{st} supernatant | 0.35 |
| 1^{st} wash | 0.34 |
| 2^{nd} wash | 0.27 |
| 2^{nd} residual cake | 0.25 |

The residual cake after five washes contained 0.48 percent astaxanthin by total weight. This was equivalent to an astaxanthin yield of 80 percent by total weight. In order to increase the astaxanthin yield, the cake was re-suspended with oil and a three-step counter-current centrifugation process was applied, with the last cake wash using fresh rice bran oil. The final astaxanthin concentration of the cake after this second counter-current extraction series was 0.25 percent by total weight, equivalent to a final astaxanthin yield of 90 percent by weight.

8. The carotenoid-enriched edible solvent collected from the centrifugation process (Example 5) was “polish filtered” to remove remaining suspended particulates, including microbial cells. Polish filtration is a process by which the carotenoid-enriched edible solvent and its residual particulate load is filtered through a centrifuge cake consisting of residual solids. This polish filtration was conducted using the same Robatel centrifuge equipped with the perforated-bowl as in previous examples. A pre-coat consisting of a suspension of 1 kilograms diatomaceous earth (Hyflo™) in 8 kilograms of fresh rice bran oil was applied to the 50 micrometer filter bag. The supernatant from the pre-coat filtration was re-circulated to the centrifuge five times in order to build a centrifuge cake before conducting the polish filtration of the carotenoid-enriched edible solvent from previous extractions.

The carotenoid-enriched edible solvent from Example 5 was admixed with 4 percent by weight of diatomaceous earth as a filtration aid and transferred to the centrifuge in incremental doses to avoiding cake drying. The collected supernatant was recycled three times back centrifuge, with the final supernatant collected aseptically to a storage container. All steps of this process were conducted at temperatures of about 25 degrees Celsius. The initial aerobic bacteria count before the polish filtration was 500 colony-forming units per gram (CFU/g). The first polish filtration reduced the bacterial load to 50 CFU/g, and this decreased to zero CFU/g after the second polish filtration.

9. Astaxanthin, like many other carotenoids, degrades in the presence of light and oxygen. Elavated temperatures result in lower viscosity of edible solvents such as vegetable oil. Lower viscosity can aid in the transfer of astaxanthin from the algal meal to the edible solvent. The following experiment was conducted to evaluate the impact of temperature and time on the astaxanthin concentration.

Astaxanthin-enriched rice bran oil was heated in polypropylene test tubes immersed in a water bath at 70, 80, or 90 degrees Celsius. Samples were taken in time intervals shown in Table 6. Astaxanthin losses were small at 70 degrees Celsius. Increasing astaxanthin losses were observed at 80 and particularly at 90 degrees Celsius.

TABLE 6
Astaxanthin concentration in rice bran oil extract
percent of total weight).

| | Astaxanthin | Astaxanthin | Astaxanthin |
|---|---|---|---|
| Time | concentration | concentration | concentration |
| (minutes) | 70 degrees | 80 degrees | 90 degrees |
| Temperature | Celsius | Celsius | Celsius |
| 0 | 1.25 | 1.31 | 1.31 |
| 15 | | 1.32 | 1.23 |
| 33 | | 1.26 | 1.27 |
| 45 | 1.26 | | |
| 50 | | | 1.21 |
| 60 | | 1.28 | |
| 80 | 1.23 | | |
| 201 | | 1.27 | 1.21 |
| 200 | | 1.24 | 1.18 |
| 335 | 1.21 | | |
| 336 | 1.23 | | |

While the present invention has been disclosed in connection with the presently preferred embodiments described herein, it should be understood that there may be other embodiments which fall within the spirit and scope of the invention as defined by the claims. Accordingly, no limitations are to be implied or inferred in this invention except as specifically and explicitly set forth in the claims.

Industrial Applicability

This invention can be used whenever it is desired to produce carotenoid-rich edible extracts from microorganisms without the use of non-edible solvents.

What is claimed is:

1. A process for producing carotenoid-rich edible extracts from a carotenoid-containing starting material, comprising:
bringing an edible solvent into direct contact with the carotenoid-containing starting material for a period of time sufficient to effectuate the transfer of at least a fraction of the carotenoid from said carotenoid-containing starting material to the edible solvent;
separating said edible solvent from said carotenoid-containing starting material using at least one mechanical process, whereby a carotenoid-enriched edible extract and a residual solid are produced.

2. A process according to claim 1, wherein said carotenoid-containing starting material is chosen from microorganisms consisting of microalgae, bacteria, cyanobacteria, fungi, yeasts, mixtures thereof, products derived from such microorganisms, and products derived from their genes.

3. A process according to claim 1, wherein at least one mechanical aid is used to improve extraction efficiency.

4. A process according to claim 1, wherein said mechanical process is chosen from the group consisting of centrifugation, pressure, and vacuum.

5. A process according to claim 3, wherein at least one mechanical aid is selected from the group consisting of centrifuges, perforated-bowl centrifuges, solid bowl centrifuges, membrane filter presses, vacuum filters, and vacuum presses.

6. A process according to claim 1, wherein the separation step comprises applying centrifugal forces of at least 100 times the force of gravity.

7. A process according to claim 1, wherein the separation step comprises applying centrifugal forces preferably in the range of 300-6000 times the force of gravity.

8. A process according to claim 1, wherein the separation step comprises applying pressures of at least 20 pounds per square inch.

9. A process according to claim 1, wherein the separation step comprises applying pressures between approximately 50 and approximately 600 pounds per square inch.

10. A process according to claim 1, wherein the separation step comprises applying vacuum in the range of 75 to 250 Torrs.

11. A process according to claim 1, wherein the separation step comprises applying vacuum of approximately 200 Torrs.

12. A process according to claim 1, wherein said carotenoid-containing starting material is subjected to a counter-current extraction process involving a plurality of mixing and separation stages, wherein the carotenoid-enriched edible extract from each separation stage is returned to the previous, or other, mixing stages, concluding with separating the final carotenoid-enriched edible extract from the last residual solid

13. A process according to claim 1, wherein edible solvent is selected from the group consisting of vegetable oils, mono-, di-, and tri-glycerides, lecithin, hydrogenated or partially hydrated fats and oils, edible animal fats and oils, and mixtures thereof.

14. A process according to claim 1, wherein said residual solids form a cake comprising the original carotenoid-containing particulate solids derived from microorganisms, and, as required, a certain quantity of a suitable filtration aid is formed.

15. A process according to claim 1, wherein said residual solids form a cake, and further comprising filtering said edible solvent through said cake to extract carotenoids.

16. A process according to claim 1, wherein said residual solids form a cake, further comprising filtering said edible solvent through said cake to reduce the amount of particulates in the edible solvent.

17. A process according to claim 1, wherein said residual solids form a cake, further comprising filtering said edible solvent through said cake to reduce microorganism contamination in the edible solvent.

18. A process according to claim 1, wherein said separation step is carried out at temperatures between approximately 0 and approximately 60 degrees Celsius.

19. A process according to claim 1, wherein said separation step is carried out at temperatures of at least approximately 20 degrees Celsius

20. A process according to claim 1, wherein said separation step is carried out at temperatures between approximately 40 and approximately 70 degrees Celsius.

21. A process according to claim 1, wherein said carotenoid-containing starting material has a moisture content less than approximately 90 percent by weight.

22. A process according to claim 1, wherein said carotenoid-containing starting material has a moisture content less than approximately 10 percent by weight.

23. A process according to claim 1, wherein said carotenoid-containing starting material has a moisture content of less than approximately 5 percent by weight.

24. A process according to claim 1, wherein the weight of the edible solvent is approximately 30 percent to approximately 150 percent by weight of the carotenoid-containing starting material.

25. A process according to claim 1, wherein the weight of the edible solvent is between approximately 75 percent and approximately 120 percent by weight of the carotenoid-containing starting material.

26. A process according to claim 1, wherein the weight of the edible solvent is between approximately 85 percent and approximately 105 percent by weight of the carotenoid-containing starting material.

27. A process according to claim 1, wherein the carotenoid is selected from the group consisting of carotenes, xanthophylls, their derivatives, and mixtures thereof.

28. A process according to claim 1, wherein the carotenoid is astaxanthin.

29. A process according to claim 1, wherein the carotenoid is an astaxanthin derivative.

30. A process according to claim 1, wherein the carotenoid-containing starting material is treated by any one or more of the physical or biochemical means selected from the group consisting of a tissue grinder, a sonicator, a homogenizer, a bead mill, a hammer mill, a French press, a cellular disruptor, a Jameson cell, a blender, pressure differentials, osmotic shock, and enzymes.

31. A process according to claim 1, further comprising adding water to the mixture of carotenoid-containing starting material and edible solvent to aid in the separation and extraction.

32. A process according to claim 1, further comprising adding filtration aids selected from the group consisting of diatomaceous earth, cellulose fiber, bentonite and similar materials to modify the characteristics of the extraction mixture.

33. A process according to claim 1, further comprising adding an inert gas to reduce the oxidation of the mixture of carotenoid-containing starting material and edible solvent.

34. A process according to claim 1, further comprising including said final carotenoid-enriched edible extract in the formulation of the group consisting of human dietary supplements, cosmetics, pharmaceuticals, coloring agents, and foodstuffs, and animal supplements, nutrition products, and animal care products
